(19) United States
(12) Patent Application Publication
(10) Pub. No.: US 2025/0345630 A1
(43) Pub. Date: Nov. 13, 2025

(54) DEEP LEARNING-BASED MULTILEAF COLLIMATOR LEAF SEQUENCING FOR MRI-GUIDED ONLINE ADAPTIVE RADIOTHERAPY

(71) Applicants: Ahmet Efe Ahunbay, Madison, WI (US); The Medical College of Wisconsin, Inc., Milwaukee, WI (US)

(72) Inventors: Ahmet Efe Ahunbay, Madison, WI (US); Ying Zhang, Brookfield, WI (US); Ergun Emin Ahunbay, New Berlin, WI (US)

(21) Appl. No.: 19/201,567

(22) Filed: May 7, 2025

Related U.S. Application Data
(60) Provisional application No. 63/643,556, filed on May 7, 2024.

(51) Int. Cl. A61N 5/10

(52) U.S. Cl. CPC A61N 5/1036; A61N 5/1039; A61N 5/1045; A61N 5/1071

(57) ABSTRACT

An optimized deliverable leaf sequence for a multileaf collimator (MLC) is rapidly generated using a deep learning model. A fluence map is input to the deep learning model, generating field segment shapes for the MLC as an output. The deep learning model may be a generative adversarial network (GAN), such as a conditional GAN. Monitor unit weights are generated for each segment in the field segment shapes. The field segment shapes and monitor unit weights may be output as the deliverable leaf sequence for the MLC.

### CROSS-REFERENCE TO RELATED APPLICATIONS

This application claims the benefit of U.S. Provisional Patent Application Ser. No. 63/643,556, filed on May 7, 2024, and entitled “DEEP LEARNING-BASED MULTILEAF COLLIMATOR LEAF SEQUENCING FOR MRI-GUIDED ONLINE ADAPTIVE RADIOTHERAPY,” which is herein incorporated by reference in its entirety.

### BACKGROUND

Fast and automated plan generation is desirable in radiation therapy to reduce planning time. One of the bottlenecks for magnetic resonance (MR)-guided online adaptive radiation therapy (MRgOART) is the time-consuming daily online replanning process. The daily replanning process for MRgOART includes two phases. In the first phase, an initial dose distribution is generated based on given dosimetric criteria. In the second phase, the final deliverable plan with optimized leaf sequences for the multileaf collimator (MLC) is generated.

With current technologies, the MRgOART process can take between 30-90 minutes. A contributing factor to this lengthy process is the aforementioned plan generation time, which can take up to 20 minutes. This labor intensive and time-consuming replanning process limits the regular clinical use of MRgOART.

### SUMMARY OF THE DISCLOSURE

It is an aspect of the present disclosure to provide a method for generating a multileaf collimator leaf sequence for use in a radiation treatment plan. The method includes accessing a fluence map and a deep learning model with the computer system. The deep learning model has been trained on training data to synthesize field segment shapes from input fluence maps. The fluence map is input to the deep learning model using the computer system, generating field segment shape data as an output. Monitor unit weights are generated for each segment in the field segment shape data using the computer system. The field segment shape data and monitor unit weights are then output with the computer system as the multileaf collimator leaf sequence. Other embodiments of this aspect include corresponding systems (e.g., computer systems), programs, algorithms, and/or modules, each configured to perform the steps of the methods.

It is another aspect of the present disclosure to provide a method for training a generative adversarial network to synthesize field segment shapes for use with a multileaf collimator. The method includes accessing training data with a computer system, where the training data include fluence map data and ground truth field segment shape data. The fluence map data may include at least one fluence map. The method also includes accessing a generative adversarial network with the computer system, where the generative adversarial network includes a generator network and a discriminator network. The generative adversarial network is trained on the training data by inputting the fluence map data to the generator network, inputting the fluence map data and the ground truth field segment shape data to the discriminator network, and minimizing a first loss for the generator network and a second a loss for the discriminator network. The trained generative adversarial network may then be stored with the computer system. Other embodiments of this aspect include corresponding systems (e.g., computer systems), programs, algorithms, and/or modules, each configured to perform the steps of the methods.

It is yet another aspect of the present disclosure to provide a computer-implemented method for generating a radiation treatment plan for a radiation treatment system having a multileaf collimator. The method includes receiving an initial radiation treatment plan with a computer system. A fluence map is generated from a dose distribution in the initial radiation treatment plan. Field segment shapes for the MLC of the radiation treatment system are then generated using a trained deep learning model, where the trained deep learning model receives the fluence map as an input and generates the field segment shapes as an output. Monitor unit weights are then generated for each field segment. The field segment shapes and monitor unit weights are stored as a leaf sequence for the MLC of the radiation treatment system, and an updated radiation treatment plan is generated by incorporating the leaf sequence for the MLC of the radiation treatment system into the initial radiation treatment plan. The method also includes outputting the updated radiation treatment plan with the computer system.

### BRIEF DESCRIPTION OF THE DRAWINGS

FIG. 1 is a flowchart of an example method for generating a radiation treatment plan based on a multileaf collimator leaf sequence generated using a deep learning model.

FIG. 2 is a flowchart of an example method for generating field segment shapes using a deep learning model.

FIG. 3 is a flowchart of an example method for training a deep learning model to synthesize field segment shapes for use with a multileaf collimator.

FIG. 4 is an example workflow diagram illustrating a process that includes segments shape prediction and segment weight generation using the techniques described in the present disclosure.

FIG. 5 is a block diagram of an example system for generating a multileaf collimator leaf sequence using one or more suitably trained deep learning models.

FIG. 6 is a block diagram of example components that can implement the system of FIG. 5.

### DETAILED DESCRIPTION

Described here systems and methods for generating an optimized deliverable leaf sequence for a multileaf collimator (MLC) in a radiation therapy system. To expedite the online replanning process, the disclosed systems and methods replace the time-consuming leaf sequencing process with a fast deep learning based method that can provide almost instantaneous deliverable segments, and can both speed up and improve the online optimization process.

In general, the leaf sequence is quickly and accurately generated from the planned dose or fluence. Advantageously, the leaf sequence can be generated with a reduced number of segments relative to leaf sequences generated using other means, which can reduce the delivery time and is therefore beneficial to techniques such as online adaptive radiotherapy. The disclosed systems and methods also enable real-time, beam-wise leaf sequencing that can be seamlessly integrated into an online replanning workflow, thereby significantly accelerating the daily plan adaptation for adaptive online radiotherapy. As an example, the disclosed deep learning-based methods are much faster (e.g., less than 5 s) than traditional MLC leaf sequencing methods (up to 10 minutes). This speed improvement results also in dosimetric improvements, especially for online adaptive or real-time planning software.

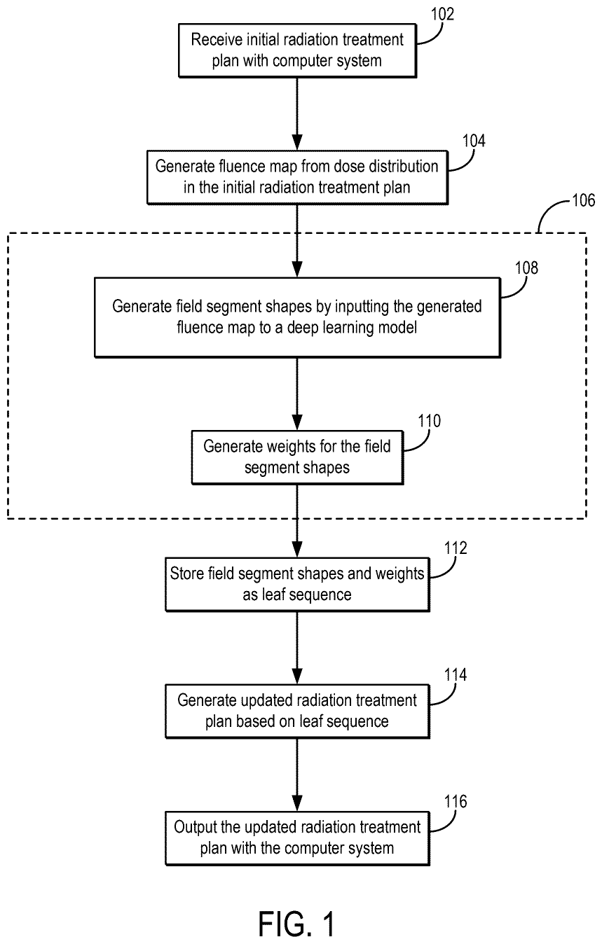

Referring now to FIG. 1, a flowchart is illustrated as setting forth the steps of an example method for generating an MLC leaf sequence for use in a radiation treatment plan. In some embodiments, the MLC leaf sequence is generated for offline or online replanning of an initial radiation treatment plan. In these instances, the method includes receiving an initial radiation treatment plan with a computer system, as indicated at step 102. When available, the initial radiation treatment plan generally includes a dose distribution. In some instances, the initial radiation treatment plan may include an initial leaf sequence for the MLC. The initial leaf sequence may include, for example, initial field segment shapes and initial weights (e.g., monitor units (MUs)). The initial radiation treatment plan may be based on simulation (e.g., reference) images, based on the daily image from a previous fraction, or otherwise be generated using a treatment planning system.

From the dose distribution, a fluence map is generated with the computer system, as indicated at step 104. As a non-limiting example, the fluence map may be generated from the dose distribution using a suitably trained deep learning model. For instance, a generative adversarial network (GAN), may be used to generate the fluence map from the dose distribution. In some implementations, a conditional GAN may be used to generate the fluence map from the dose distribution. Additionally or alternatively, the fluence map may be generated from the dose distribution using other techniques, such as using a treatment planning system. In some alternative implementations, the fluence map may be received as part of the initial radiation treatment plan. In these instances, step 104 may be omitted, and the initial fluence map may be used in the subsequent steps for generating the updated radiation treatment plan.

A leaf sequence for the MLC is then generated based on the fluence map, as indicated at process block 106. First, field segment shapes are generated, as indicated at step 108. As a non-limiting example, the field segment shapes can be generated using a suitably trained deep learning model. The deep learning model may be a transformer network, a GAN, a conditional GAN, or the like. The fluence map may be input to the deep learning model, generating field segment shapes as an output. Second, weights (e.g., MUs) for each field segment are generated, as indicated at step 110. As an example, the weights can be generated using a linear matrix equation.

The field segment shapes and weights can then be stored as the leaf sequence, as indicated at step 112. Using the leaf sequence, an updated radiation treatment plan is then generated, as indicated at step 114. For example, the updated radiation treatment plan can be generated using an adaptive online radiation therapy planning workflow that incorporates the leaf sequence into the replanning process. For instance, the deliverable leaf sequence generated for each beam can be imported into a treatment planning system for dose calculation. As a non-limiting example, to convert field segment shapes to MLC control points, the predicted field segments can be overlaid on a grid representing the maximum field size of the radiation therapy system at isocenter. For radiation therapy systems that implement beam shaping that allows for leaf movement along the y-axis and diaphragm movement along the x-axis, the x-axis of the predicted field segment can be resampled to the projected leaf width at the machine isocenter. The edges of the field segment shape along the x-axis and the y-axis can be detected and the corresponding grid coordinates found. An intensity threshold (e.g., a 2% intensity threshold) can be applied to the predicted field segment to reduce low-intensity noise. The monitor unit (MU) weights for each field segment can be assigned based on a linear matrix equation with generated segment shapes and a goal fluence map.

The updated radiation treatment plan is then output with the computer system, as indicated at step 116. Outputting the updated radiation treatment plan may include storing the updated radiation treatment plan for later use, sending the updated radiation treatment plan to a treatment planning system to control operation of a radiation therapy system, or the like.

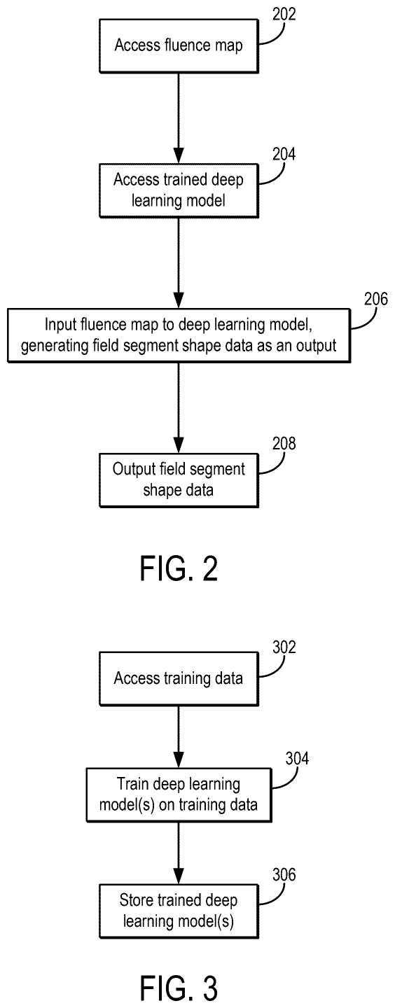

Referring now to FIG. 2, a flowchart is illustrated as setting forth the steps of an example method for generating a leaf sequence using a suitably trained deep learning model or other machine learning algorithm. As will be described, the deep learning model takes a fluence map as input data and generates field segment shape data as output data.

The method includes accessing an fluence map with the computer system, as indicated at step 202. As described above, accessing the fluence map may include retrieving such data from a memory or other suitable data storage device or medium. For example, the fluence map may be received as part of the initial radiation treatment plan. Additionally or alternatively, accessing the fluence map may include generating the fluence map from a dose distribution received as part of the initial radiation treatment plan and transferring or otherwise communicating the fluence map to the computer system. As described above, in some implementations the fluence map may be generated by inputting the dose distribution from the initial radiation treatment plan to a suitably trained deep learning model. The deep learning model may be a transformer network, a GAN, a conditional GAN, or the like.

A trained deep learning model (or other suitable machine learning algorithm) is then accessed with the computer system, as indicated at step 204. In general, the deep learning model is trained, or has been trained, on training data in order to generate field segment shapes from a fluence map.

Accessing the trained deep learning model may include accessing model parameters (e.g., weights, biases, or both) that have been optimized or otherwise estimated by training the deep learning model on training data. In some instances, retrieving the deep learning model can also include retrieving, constructing, or otherwise accessing the particular model architecture to be implemented. For instance, data pertaining to the layers in the neural network architecture (e.g., number of layers, type of layers, ordering of layers, connections between layers, hyperparameters for layers) may be retrieved, selected, constructed, or otherwise accessed.

As described above, in some implementations the deep learning model may be a GAN. In general, a GAN includes two neural networks: a discriminator network and a generator network. An artificial neural network generally includes an input layer, one or more hidden layers (or nodes), and an output layer. Typically, the input layer includes as many nodes as inputs provided to the artificial neural network. The number (and the type) of inputs provided to the artificial neural network may vary based on the particular task for the artificial neural network.

The input layer connects to one or more hidden layers. The number of hidden layers varies and may depend on the particular task for the artificial neural network. Additionally, each hidden layer may have a different number of nodes and may be connected to the next layer differently. For example, each node of the input layer may be connected to each node of the first hidden layer. The connection between each node of the input layer and each node of the first hidden layer may be assigned a weight parameter. Additionally, each node of the neural network may also be assigned a bias value. In some configurations, each node of the first hidden layer may not be connected to each node of the second hidden layer. That is, there may be some nodes of the first hidden layer that are not connected to all of the nodes of the second hidden layer. The connections between the nodes of the first hidden layers and the second hidden layers are each assigned different weight parameters. Each node of the hidden layer is generally associated with an activation function. The activation function defines how the hidden layer is to process the input received from the input layer or from a previous input or hidden layer. These activation functions may vary and be based on the type of task associated with the artificial neural network and also on the specific type of hidden layer implemented.

Each hidden layer may perform a different function. For example, some hidden layers can be convolutional hidden layers which can, in some instances, reduce the dimensionality of the inputs. Other hidden layers can perform statistical functions such as max pooling, which may reduce a group of inputs to the maximum value; an averaging layer; batch normalization; and other such functions. In some of the hidden layers each node is connected to each node of the next hidden layer, which may be referred to then as dense layers. Some neural networks including more than, for example, three hidden layers may be considered deep neural networks.

The last hidden layer in the artificial neural network is connected to the output layer. Similar to the input layer, the output layer typically has the same number of nodes as the possible outputs.

The fluence map is then input to the deep learning model, generating field segment shape data as an output, as indicated at step 208. For example, when the deep learning model is a GAN (e.g., a conditional GAN), the fluence map may be input to the generator network, generating individual field segment shapes as an output.

The field segment shape data can then be output by the computer system, such as by displaying the field segment shape data to a user, storing the field segment shape data for later use or further processing, or both, as indicated at step 210. For example, as described above the field segment shape data may be used to generate weights (e.g., MUs) for each field segment and to subsequently generate a radiation treatment plan, or an updated radiation treatment plan through a replanning process.

Referring now to FIG. 3, a flowchart is illustrated as setting forth the steps of an example method for training one or more deep learning models (or other suitable machine learning algorithms) on training data, such that the one or more deep learning models are trained to receive a fluence map as input data in order to generate field segment shape data as output data.

In general, the deep learning model can implement any number of different deep learning model architectures. As one example, the deep learning model may be a GAN, such as a conditional GAN. As another example, the deep learning model may be a transformer network or model. In still other examples, the deep learning model may otherwise implement one or more neural network architectures. For instance, the neural network(s) could implement a convolutional neural network, a residual neural network, or the like.

The method includes accessing training data with a computer system, as indicated at step 302. Accessing the training data may include retrieving such data from a memory or other suitable data storage device or medium. In general, the training data can include field segment shapes and fluence maps. Additionally, the training data may include other data, such as dose distributions (e.g., dose maps). The field segment shape data may include field segment shape data received as part of one or more radiation treatment plans, which may be generated based on reference images or daily images, from previous fractions, or the like.

The method can include assembling training data from field segment shapes, fluence maps, and any other relevant data using a computer system. This step may include assembling the field segment shapes and fluence maps into an appropriate data structure on which the neural network or other machine learning algorithm can be trained.

One or more deep learning models (or other suitable machine learning algorithms) are then trained on the training data, as indicated at step 304. In general, the deep learning model can be trained by optimizing model parameters (e.g., weights, biases, or both) based on minimizing one or more loss functions. As one non-limiting example, the deep learning model may be a GAN (e.g., a conditional GAN or other GAN architecture), which as described above is composed of a generator (G) network and a discriminator (D) network. In some implementations, the loss function used for training a GAN (e.g., a conditional GAN) can be:

$${L}_{cGAN} ( G , D ) = {\sum }_{x , y} ( log D ( x , y ) ) + {\sum }_{x , y} ( log ( 1 - D ( x , G ( x ) ) ) ) ; \qquad ( 1 )$$

During training, the generator and discriminator update their weights one at a time in an adversarial manner, such that the discriminator is trained to maximize the log probability of real images, D(x,y), and the log of the inverse probability of synthetic images, 1-D(x,G(x)). In this process, the discriminator is trained to detect the synthetic images. On the other hand, the generator is trained to minimize the loss (e.g., an L1 loss) between the synthetic images and ground truth images. Training is complete when an equilibrium is reached between the generator and discriminator losses. The final objective function can thus be defined as:

$$arg {min}_{G} {max}_{D} \{ {L}_{cGAN} ( G , D ) + λ {L}_{ℓ1} ( G ) \} ; \qquad ( 2 )$$

where λ is a hyperparameter that gives weight to the L1 loss of the generator.

As a non-limiting example, the generator architecture can be based on a convolutional neural network architecture such as a UNet architecture. For instance, the generator may include downsampling blocks that each include a convolution, batch normalization, and leaky ReLU activation function. The generator may also include upsampling blocks that each include convolutions, batch normalization, and a ReLU activation function. Skip connections between the downsampling and upsampling blocks encourage the learning of both high-level and low-level features. In some implementations, random dropout can be used in the generator to introduce noise and prevent over-fitting of the data.

The deep learning model is designed to recursively predict each field segment shape using previously predicted segments and an input fluence map. Considering the uncertainty regarding the number of segments for each beam, multiple models can be trained for varying numbers of segments per beam (ranging from 4-6) and the final plan with smallest mean-absolute-error (MAE) with the target fluence can be selected as the trained deep learning model.

The input and output of the generator were the fluence map and individual field segments, respectively. The input to the discriminator was the fluence map paired with either the ground truth field segments or predicted field segments. While the generator penalized predicted field segments different from the ground truth, the discriminator was trained to classify N×N patches of the generator output as either synthetic or real data. As one non-limiting example, the discriminator can be trained using 70×70 patches.

The one or more trained deep learning models are then stored for later use, as indicated at step 306. Storing the deep learning model(s) may include storing model parameters (e.g., weights, biases, or both), which have been computed or otherwise estimated by training the deep learning model(s) on the training data. Storing the trained deep learning model(s) may also include storing the particular model architecture to be implemented. For instance, data pertaining to the layers in the neural network architecture (e.g., number of layers, type of layers, ordering of layers, connections between layers, hyperparameters for layers) may be stored.

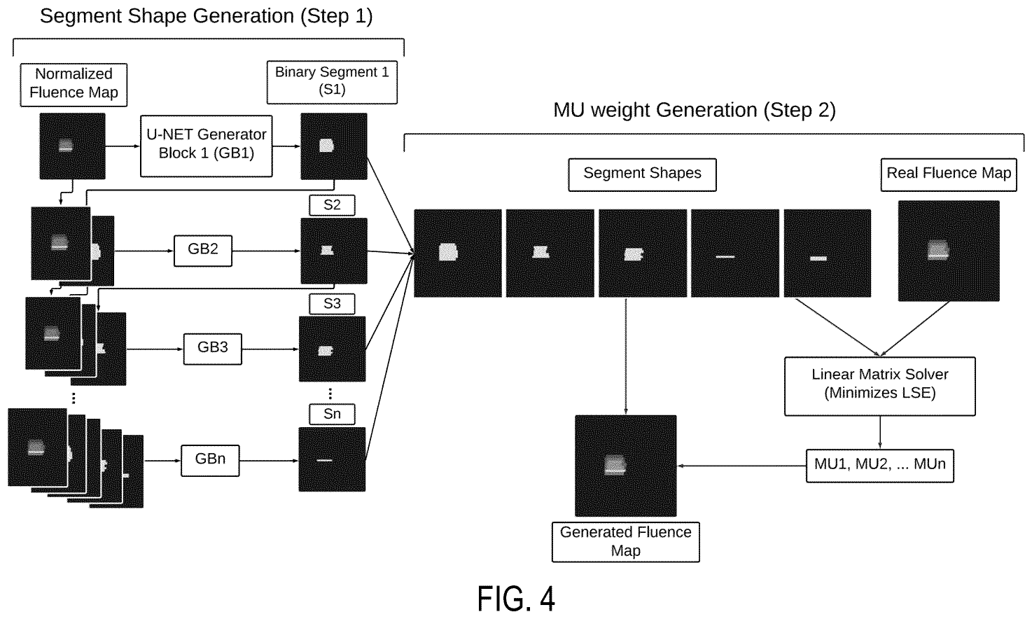

FIG. 4 illustrates an example process for generating segment shapes and MU weights using the method described in the present disclosure. The example workflow includes two stages. The first stage uses a recurrent conditional GAN (rcGAN) model to predict binary segment shapes from an input fluence map, where the shape of each segment is predicted recurrently using the previously predicted segments and the fluence maps as inputs. The second stage uses a linear matrix equation module to generate the MU weights for the segments.

The backbone of the segment shape prediction network is a cGAN with modifications. An rcGAN architecture may be used to iteratively generate segment shapes through N unique blocks, where N is the predefined number of segments per beam. A customized loss function, L_{sum_mae}, which can be defined as the mean absolute error (MAE) between the reconstructed fluence map (FM_{pred})—the summation of N generated segments—and the original fluence map (FM_{org}). This loss minimizes the overall fluence map difference, rather than focusing on the differences in individual segments. The final objective function, F, may be defined as:

$$F = arg {min}_{G} {max}_{D} {L}_{cGAN} ( G , D ) + λ {L}_{sum \_ mae} ( G ) ; \qquad ( 3 )$$

where L_{sum_mae}=|FM_{org}−FM_{pred}, L_{cGAN} is the loss function from Eqn. (1), and λ is a hyperparameter that gives weight to the loss of the generator. Once all the segment shapes are determined, the MU values for each segment may be calculated by solving a linear matrix equation using least-squares solution:

$$MU = {( {Segs}^{T} Segs )}^{- 1} {Segs}^{T} \cdot {FM}_{org} ; \qquad ( 4 )$$

where Segs is a matrix that represents the shapes of the beam segments and is of size (S_{f}, N_{seg}), where S_{f} is the flattened segment shape vector (essentially converting the two-dimensional shape of the beam's segment into a one-dimensional array), and N_{seg} is the total number of segments of the beam.

To allow for flexible segment predictions and explore the impact of different number of segments, DL models were trained with N=4, 5, 6, and 7 segments per beam. For the training data of each model, N segments were selected to reconstruct the FM. Beams with fewer than N segments were excluded, while beams with more segments used random combinations to create multiple fake training FM. Data augmentation was performed through random segment ordering and flipping in the x and y directions. All the FMs were then normalized to [0, 1] for DL model training and testing purpose.

For testing, for a given test FM, the leaf sequence was first generated using each of the trained models (with 4-7 segments). Then, the relative absolute error (rAE) compared to the original FM was calculated for each leaf sequence, and the one with the smallest rAE was selected. The rAE was defined as the ratio of the sum of absolute errors to the sum of the original input FM.

The example described above demonstrated the DL-based approach aimed at replacing the time-consuming iterative optimization-based leaf sequencing algorithm in current treatment planning systems for MRoART or other radiation therapy techniques. The methods described in the present disclosure enable the rapid and accurate generation of deliverable MLC sequences from a fluence map, which is advantageous for online plan adaptation. This method can produce final deliverable leaf sequences with fewer segments, reducing plan delivery time while maintaining the same plan quality, which is a significant advantage for enhancing patient comfort and optimizing valuable machine time.

The methods described above presented a general approach for converting a 2D fluence maps into multiple MLC segments with corresponding weights using a deep learning method. These methods are adaptable because the input is a beam-wise fluence map and is not restricted to any specific tumor site or machine. Advantageously, the method have the potential to be implemented across various anatomical treatment sites and extended to step and shot (S&S) IMRT plans on regular Linac treatment plans and/ot VMAT-type treatment plans.

In the examples describes above, a two-step method was used to generate the segment shape and MU weights separately. In some other cases, joint optimization or iterative feedback mechanisms can be used to achieve a more global optimum and to jointly generate the segment shapes and MU weights.

Advantageously, the methods described in the present disclosure can be used in conjunction with DL-based dose and fluence map prediction techniques using the daily anatomy/image. In these cases, the disclosed methods can convert the predicted fluence maps into machine-deliverable plans. Moreover, the methods can enable the capability of interactive fluence map-based plan adjustment to accept human interventions, overcoming the limitation of hard-to-adjust dose distribution in DL-based dose prediction, such as lowering certain OAR doses for patient-specific planning. Advantageously, this approach can be useful for plan updates due to the small changes during real-time delivery. The manipulated fluence map can be quickly converted to an MLC leaf sequence and to get the updated dose distribution. Such a proposed pipeline allows for more streamlined and efficient workflows, enabling online or real-time adaption of treatment plans.

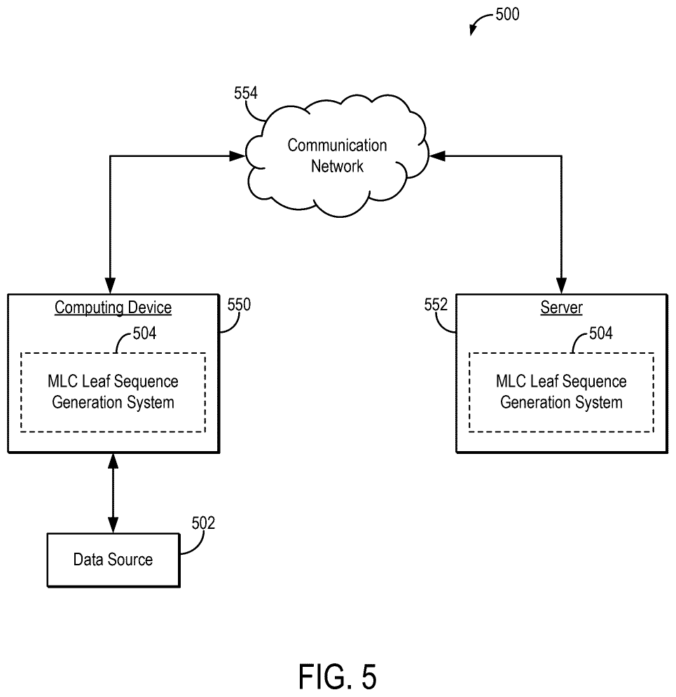

FIG. 5 shows an example of a system 500 for generating an MLC leaf sequence for use in a radiation treatment plan in accordance with some embodiments described in the present disclosure. As shown in FIG. 5, a computing device 550 can receive one or more types of data (e.g., initial radiation treatment plan, a dose distribution, a fluence map, an initial leaf sequence, initial field segment shape data, daily images, etc.) from data source 502. In some embodiments, computing device 550 can execute at least a portion of an MLC leaf sequence generation system 504 to generate an MLC leaf sequence from data received from the data source 502.

Additionally or alternatively, in some embodiments, the computing device 550 can communicate information about data received from the data source 502 to a server 552 over a communication network 554, which can execute at least a portion of the MLC leaf sequence generation system 504. In such embodiments, the server 552 can return information to the computing device 550 (and/or any other suitable computing device) indicative of an output of the MLC leaf sequence generation system 504.

In some embodiments, computing device 550 and/or server 552 can be any suitable computing device or combination of devices, such as a desktop computer, a laptop computer, a smartphone, a tablet computer, a wearable computer, a server computer, a virtual machine being executed by a physical computing device, and so on.

In some embodiments, data source 502 can be any suitable source of data (e.g., initial radiation treatment plan, a dose distribution, a fluence map, an initial leaf sequence, initial field segment shape data, etc.), such as a radiation treatment planning system, another computing device (e.g., a server storing initial radiation treatment plan, a dose distribution, a fluence map, an initial leaf sequence, initial field segment shape data, etc.), and so on. In some embodiments, data source 502 can be local to computing device 550. For example, data source 502 can be incorporated with computing device 550 (e.g., computing device 550 can be configured as part of a device for measuring, recording, estimating, acquiring, or otherwise collecting or storing data). As another example, data source 502 can be connected to computing device 550 by a cable, a direct wireless link, and so on. Additionally or alternatively, in some embodiments, data source 502 can be located locally and/or remotely from computing device 550, and can communicate data to computing device 550 (and/or server 552) via a communication network (e.g., communication network 554).

In some embodiments, communication network 554 can be any suitable communication network or combination of communication networks. For example, communication network 554 can include a Wi-Fi network (which can include one or more wireless routers, one or more switches, etc.), a peer-to-peer network (e.g., a Bluetooth network), a cellular network (e.g., a 3G network, a 4G network, etc., complying with any suitable standard, such as CDMA, GSM, LTE, LTE Advanced, WiMAX, etc.), other types of wireless network, a wired network, and so on. In some embodiments, communication network 554 can be a local area network, a wide area network, a public network (e.g., the Internet), a private or semi-private network (e.g., a corporate or university intranet), any other suitable type of network, or any suitable combination of networks. Communications links shown in FIG. 5 can each be any suitable communications link or combination of communications links, such as wired links, fiber optic links, Wi-Fi links, Bluetooth links, cellular links, and so on.

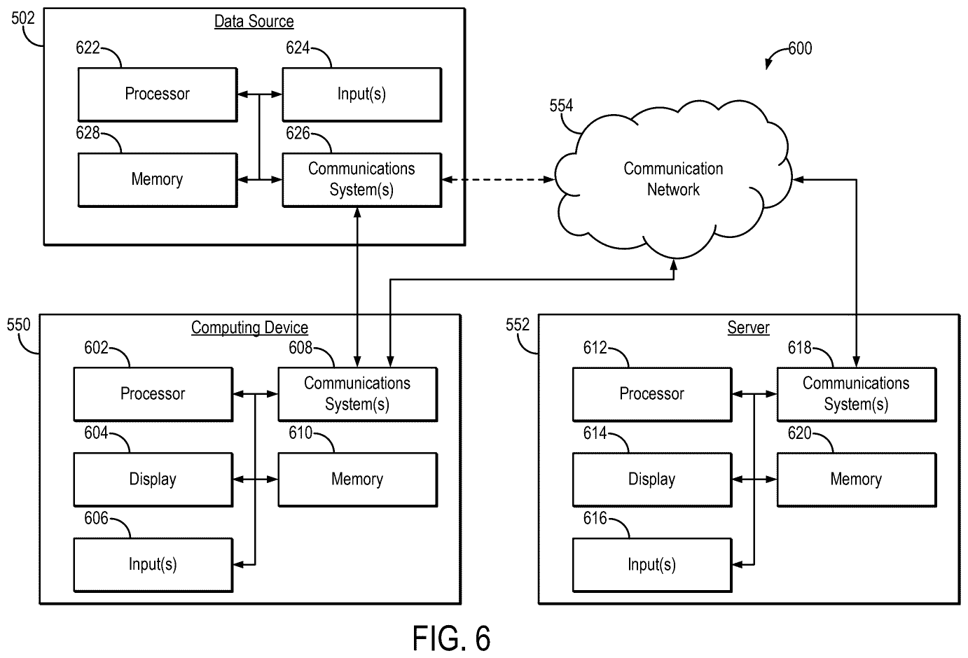

Referring now to FIG. 6, an example of hardware 600 that can be used to implement data source 502, computing device 550, and server 552 in accordance with some embodiments of the systems and methods described in the present disclosure is shown.

As shown in FIG. 6, in some embodiments, computing device 550 can include a processor 602, a display 604, one or more inputs 606, one or more communication systems 608, and/or memory 610. In some embodiments, processor 602 can be any suitable hardware processor or combination of processors, such as a central processing unit (CPU), a graphics processing unit (GPU), and so on. In some embodiments, display 604 can include any suitable display devices, such as a liquid crystal display (LCD) screen, a light-emitting diode (LED) display, an organic LED (OLED) display, an electrophoretic display (e.g., an “e-ink” display), a computer monitor, a touchscreen, a television, and so on. In some embodiments, inputs 606 can include any suitable input devices and/or sensors that can be used to receive user input, such as a keyboard, a mouse, a touchscreen, a microphone, and so on.

In some embodiments, communications systems 608 can include any suitable hardware, firmware, and/or software for communicating information over communication network 554 and/or any other suitable communication networks. For example, communications systems 608 can include one or more transceivers, one or more communication chips and/or chip sets, and so on. In a more particular example, communications systems 608 can include hardware, firmware, and/or software that can be used to establish a Wi-Fi connection, a Bluetooth connection, a cellular connection, an Ethernet connection, and so on.

In some embodiments, memory 610 can include any suitable storage device or devices that can be used to store instructions, values, data, or the like, that can be used, for example, by processor 602 to present content using display 604, to communicate with server 552 via communications system(s) 608, and so on. Memory 610 can include any suitable volatile memory, non-volatile memory, storage, or any suitable combination thereof. For example, memory 610 can include random-access memory (RAM), read-only memory (ROM), electrically programmable ROM (EPROM), electrically erasable ROM (EEPROM), other forms of volatile memory, other forms of non-volatile memory, one or more forms of semi-volatile memory, one or more flash drives, one or more hard disks, one or more solid state drives, one or more optical drives, and so on. In some embodiments, memory 610 can have encoded thereon, or otherwise stored therein, a computer program for controlling operation of computing device 550. In such embodiments, processor 602 can execute at least a portion of the computer program to present content (e.g., images, user interfaces, graphics, tables), receive content from server 552, transmit information to server 552, and so on. For example, the processor 602 and the memory 610 can be configured to perform the methods described herein (e.g., the method of FIG. 1, the method of FIG. 2, the method of FIG. 3).

In some embodiments, server 552 can include a processor 612, a display 614, one or more inputs 616, one or more communications systems 618, and/or memory 620. In some embodiments, processor 612 can be any suitable hardware processor or combination of processors, such as a CPU, a GPU, and so on. In some embodiments, display 614 can include any suitable display devices, such as an LCD screen, LED display, OLED display, electrophoretic display, a computer monitor, a touchscreen, a television, and so on. In some embodiments, inputs 616 can include any suitable input devices and/or sensors that can be used to receive user input, such as a keyboard, a mouse, a touchscreen, a microphone, and so on.

In some embodiments, communications systems 618 can include any suitable hardware, firmware, and/or software for communicating information over communication network 554 and/or any other suitable communication networks. For example, communications systems 618 can include one or more transceivers, one or more communication chips and/or chip sets, and so on. In a more particular example, communications systems 618 can include hardware, firmware, and/or software that can be used to establish a Wi-Fi connection, a Bluetooth connection, a cellular connection, an Ethernet connection, and so on.

In some embodiments, memory 620 can include any suitable storage device or devices that can be used to store instructions, values, data, or the like, that can be used, for example, by processor 612 to present content using display 614, to communicate with one or more computing devices 550, and so on. Memory 620 can include any suitable volatile memory, non-volatile memory, storage, or any suitable combination thereof. For example, memory 620 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 620 can have encoded thereon a server program for controlling operation of server 552. In such embodiments, processor 612 can execute at least a portion of the server program to transmit information and/or content (e.g., data, images, a user interface) to one or more computing devices 550, receive information and/or content from one or more computing devices 550, receive instructions from one or more devices (e.g., a personal computer, a laptop computer, a tablet computer, a smartphone), and so on.

In some embodiments, the server 552 is configured to perform the methods described in the present disclosure. For example, the processor 612 and memory 620 can be configured to perform the methods described herein (e.g., the method of FIG. 1, the method of FIG. 2, the method of FIG. 3).

In some embodiments, data source 502 can include a processor 622, one or more inputs 624, one or more communications systems 626, and/or memory 628. In some embodiments, processor 622 can be any suitable hardware processor or combination of processors, such as a CPU, a GPU, and so on. In some embodiments, the one or more inputs 624 are generally configured to acquire data, images, or both, and can include a radiation treatment planning system, an MRI system, other data sources, or combinations thereof. Additionally or alternatively, in some embodiments, the one or more inputs 624 can include any suitable hardware, firmware, and/or software for coupling to and/or controlling operations of a radiation treatment planning system, an MRI system, or the like. In some embodiments, one or more portions of the input(s) 624 can be removable and/or replaceable.

Note that, although not shown, data source 502 can include any suitable inputs and/or outputs. For example, data source 502 can include input devices and/or sensors that can be used to receive user input, such as a keyboard, a mouse, a touchscreen, a microphone, a trackpad, a trackball, and so on. As another example, data source 502 can include any suitable display devices, such as an LCD screen, an LED display, an OLED display, an electrophoretic display, a computer monitor, a touchscreen, a television, etc., one or more speakers, and so on.

In some embodiments, communications systems 626 can include any suitable hardware, firmware, and/or software for communicating information to computing device 550 (and, in some embodiments, over communication network 554 and/or any other suitable communication networks). For example, communications systems 626 can include one or more transceivers, one or more communication chips and/or chip sets, and so on. In a more particular example, communications systems 626 can include hardware, firmware, and/or software that can be used to establish a wired connection using any suitable port and/or communication standard (e.g., VGA, DVI video, USB, RS-232, etc.), Wi-Fi connection, a Bluetooth connection, a cellular connection, an Ethernet connection, and so on.

In some embodiments, memory 628 can include any suitable storage device or devices that can be used to store instructions, values, data, or the like, that can be used, for example, by processor 622 to control the one or more inputs 624, and/or receive data from the one or more inputs 624; to generate images from data; present content (e.g., data, images, a user interface) using a display; communicate with one or more computing devices 550; and so on. Memory 628 can include any suitable volatile memory, non-volatile memory, storage, or any suitable combination thereof. For example, memory 628 can include RAM, ROM, EPROM, EEPROM, other types of volatile memory, other types of non-volatile memory, one or more types of semi-volatile memory, one or more flash drives, one or more hard disks, one or more solid state drives, one or more optical drives, and so on. In some embodiments, memory 628 can have encoded thereon, or otherwise stored therein, a program for controlling operation of data source 502. In such embodiments, processor 622 can execute at least a portion of the program to generate images, transmit information and/or content (e.g., data, images, a user interface) to one or more computing devices 550, receive information and/or content from one or more computing devices 550, receive instructions from one or more devices (e.g., a personal computer, a laptop computer, a tablet computer, a smartphone, etc.), and so on.

In some embodiments, any suitable computer-readable media can be used for storing instructions for performing the functions and/or processes described herein. For example, in some embodiments, computer-readable media can be transitory or non-transitory. For example, non-transitory computer-readable media can include media such as magnetic media (e.g., hard disks, floppy disks), optical media (e.g., compact discs, digital video discs, Blu-ray discs), semiconductor media (e.g., RAM, flash memory, EPROM, EEPROM), any suitable media that is not fleeting or devoid of any semblance of permanence during transmission, and/or any suitable tangible media. As another example, transitory computer-readable media can include signals on networks, in wires, conductors, optical fibers, circuits, or any suitable media that is fleeting and devoid of any semblance of permanence during transmission, and/or any suitable intangible media.

As used herein in the context of computer implementation, unless otherwise specified or limited, the terms “component,” “system,” “module,” “framework,” and the like are intended to encompass part or all of computer-related systems that include hardware, software, a combination of hardware and software, or software in execution. For example, a component may be, but is not limited to being, a processor device, a process being executed (or executable) by a processor device, an object, an executable, a thread of execution, a computer program, or a computer. By way of illustration, both an application running on a computer and the computer can be a component. One or more components (or system, module, and so on) may reside within a process or thread of execution, may be localized on one computer, may be distributed between two or more computers or other processor devices, or may be included within another component (or system, module, and so on).

In some implementations, devices or systems disclosed herein can be utilized or installed using methods embodying aspects of the disclosure. Correspondingly, description herein of particular features, capabilities, or intended purposes of a device or system is generally intended to inherently include disclosure of a method of using such features for the intended purposes, a method of implementing such capabilities, and a method of installing disclosed (or otherwise known) components to support these purposes or capabilities. Similarly, unless otherwise indicated or limited, discussion herein of any method of manufacturing or using a particular device or system, including installing the device or system, is intended to inherently include disclosure, as embodiments of the disclosure, of the utilized features and implemented capabilities of such device or system.

The present disclosure has described one or more preferred embodiments, and it should be appreciated that many equivalents, alternatives, variations, and modifications, aside from those expressly stated, are possible and within the scope of the invention.

1. A method for generating a multileaf collimator leaf sequence for use in a radiation treatment plan, the method comprising:
accessing a fluence map with the computer system;
accessing a deep learning model with the computer system, wherein the deep learning model has been trained on training data to synthesize field segment shapes from input fluence maps;
inputting the fluence map to the deep learning model using the computer system, generating field segment shape data as an output;
generating monitor unit weights for each segment in the field segment shape data using the computer system; and
outputting, with the computer system, the field segment shape data and monitor unit weights as the multileaf collimator leaf sequence.

2. The method of claim 1, wherein the deep learning model comprises a generative adversarial network (GAN).

3. The method of claim 2, wherein the GAN comprises a conditional GAN.

4. The method of claim 1, wherein accessing the fluence map with the computer system comprises accessing a dose distribution map with the computer system and generating the fluence map from the dose distribution map.

5. The method of claim 4, wherein generating the fluence map from the dose distribution map comprises accessing a second deep learning model that has been trained on training data to synthesize fluence maps from input dose distribution maps; and inputting the dose distribution map to the second deep learning model using the computer system, generating the fluence map as an output.

6. The method of claim 1, wherein the monitor unit weights are generated for each segment in the field segment shape data using a linear matrix equation.

7. The method of claim 1, further comprising generating a radiation treatment plan using the multileaf collimator leaf sequence and outputting the radiation treatment plan to a radiation therapy system to control operation of the radiation therapy system.

8. The method of claim 7, wherein the radiation therapy system comprises an intensity modulated radiation therapy (IMRT) system.

9. The method of claim 7, wherein the radiation therapy system comprises a magnetic resonance (MR)-guided radiation therapy system.

10. The method of claim 1, wherein outputting the field segment shape data and monitor unit weights as the multileaf collimator leaf sequence comprises converting the field segment shape data to multileaf collimator control points and storing the multileaf collimator control points as part of the multileaf collimator leaf sequence.

11. A method for training a generative adversarial network to synthesize field segment shapes for use with a multileaf collimator, the method comprising:
accessing training data with a computer system, wherein the training data comprises:
fluence map data comprising at least one fluence map;
ground truth field segment shape data;
accessing a generative adversarial network with the computer system, wherein the generative adversarial network comprises a generator network and a discriminator network;
training the generative adversarial network on the training data by:
inputting the fluence map data to the generator network;
inputting the fluence map data and the ground truth field segment shape data to the discriminator network;
minimizing a first loss for the generator network and a second loss for the discriminator network; and
storing the trained generative adversarial network with the computer system.

12. The method of claim 11, wherein minimizing the first loss for the generator network comprises minimizing a loss between synthetic field segment shape data and the ground truth field segment shape data.

13. The method of claim 12, wherein the first loss for the generator network is an L1 loss.

14. The method of claim 11, wherein minimizing the second loss for the discriminator network comprises minimizing a loss between synthetic field segment shape data and the ground truth field segment shape data.

15. The method of claim 14, wherein the second loss for the discriminator network comprises a loss between a log probability of the ground truth segment shape data and an inverse probability of synthetic field segment shape data.

16. A computer-implemented method for generating a radiation treatment plan for a radiation treatment system having a multileaf collimator (MLC), comprising:
receiving an initial radiation treatment plan with a computer system;
generating a fluence map from a dose distribution in the initial radiation treatment plan;
generating field segment shapes for the MLC of the radiation treatment system using a trained deep learning model, wherein the trained deep learning model receives the fluence map as an input and generates the field segment shapes as an output;
generating monitor unit weights for each field segment;
storing the field segment shapes and monitor unit weights as a leaf sequence for the MLC;
generating an updated radiation treatment plan incorporating the leaf sequence for the MLC into the initial radiation treatment plan; and
outputting the updated radiation treatment plan with the computer system.

17. The method of claim 16, wherein generating the fluence map comprises inputting the dose distribution to a trained generative adversarial network (GAN).

18. The method of claim 16, wherein the trained deep learning model is a transformer network.

19. The method of claim 16, wherein the trained deep learning model is a generative adversarial network (GAN).

20. The method of claim 16, wherein outputting the updated radiation treatment plan with the computer system comprises controlling the radiation treatment system to deliver radiation according to the updated radiation treatment plan.
